(19) United States
(12) Patent Application Publication
(10) Pub. No.: US 2023/0381094 A1
(43) Pub. Date: Nov. 30, 2023

(54) Cosmetic Composition with Skin Microbiome-Friendly Properties

(71) Applicant: LG Household & Health Care Ltd., Seoul (KR)

(72) Inventors: Jaehyun Park, Seoul (KR); Sangah Hong, Seoul (KR); Sunghwan Hwang, Seoul (KR); Nakyung Han, Seoul (KR); Nam-Seo Son, Seoul (KR); Seung-Jin Hwang, Seoul (KR)

(73) Assignee: LG Household & Health Care Ltd., Seoul (KR)

(21) Appl. No.: 18/203,956

(22) Filed: May 31, 2023

(30) Foreign Application Priority Data
May 31, 2022 (KR) 10-2022-0066841

(51) Int. Cl. A61K 8/99; A61K 8/55; A61K 8/92; A61K 8/37; A61K 8/42; A61Q 19/00; A61Q 17/00

(52) U.S. Cl. CPC A61K 8/99; A61K 8/553; A61K 8/922; A61K 8/37; A61K 8/42; A61Q 19/00; A61Q 17/00

(57) ABSTRACT

The present invention relates to the use of a composition with beneficial skin bacteria-friendly properties. The composition of the present invention does not inhibit the growth of beneficial skin bacteria, has beneficial skin bacteria-friendly properties, can maintain or promote the growth of beneficial bacteria without addition of active materials such as prebiotics or probiotics, and therefore can be provided as a microbiome cosmetic composition.

### TECHNICAL FIELD

The present invention relates to the use of a composition with beneficial skin bacteria-friendly properties. The composition may be provided as a cosmetic.

### BACKGROUND ART

In recent years, consumers' interest in “microbiome cosmetics”, which are effective in protecting damaged or weakened skin due to external stimuli and stress such as fine dust and ultraviolet rays, has increased.

Microbiome cosmetics are cosmetics that can maintain healthy skin or improve skin conditions by inducing changes in skin microbiota through application of materials that can directly or indirectly affect the growth of the “skin flora” inhabiting human skin. According to previous studies, the skin flora plays an important role in maintaining the skin's immune system and protecting the skin from some environmental hazards including pathogens (Byrd, A. L. et al. (2018) Nature Reviews Microbiology, 16(3), 143). Therefore, by maintaining the balance between the flora present in the skin and creating an environment favorable to the growth of beneficial bacteria through studies on microbiome cosmetics, effects of strengthening skin immunity, reducing acne, improving skin barrier, and the like can be expected (S. Lebeer et al. (2018) BioRxiv. doi: doi.org/10.1101/463307).

The market need for microbiome cosmetics with these features is steadily rising, and multinational corporations (MNC) are running their own research institutes for studies on microbiome cosmetics or conducting various studies through collaboration with pharmaceutical companies. However, the current studies on microbiome cosmetics are focused on discovering active materials having efficacy of “prebiotics” or “probiotics” and applying them.

Most microbiome cosmetics that have been studied and released to date are mainly products containing 1) prebiotics (unsustainable food elements that confer benefits to the host by inducing changes in the microbiota or helping the survival environment of beneficial bacteria) and 2) probiotics (microorganisms that confer benefits to the host and maintain their population in an appropriate amount, symbiotically) in general cosmetic formulation ingredients (thickeners, emulsifiers, and ingredients required for water/inner phase stabilization), or in a slightly wider range, there are also products containing 3) postbiotics (substances produced by metabolism or fermentation of probiotics).

However, ingredients such as prebiotics, probiotics, and/or postbiotics ionize, or their chemical properties are not clear, and thus these exhibit poor compatibility with cosmetic formulation ingredients. As a result, when the ingredients are used in excess for high efficacy and high added value, formulation stability may deteriorate, and precipitation and phase separation may occur. In order to prevent this, the content of prebiotic, probiotic, and/or postbiotic ingredients in most microbiome cosmetics is inevitably insignificant.

In addition, there is no known effect of formulation ingredients that account for the majority of cosmetic compositions on the skin flora and interaction thereof with active ingredients since there have been no prior studies on the microbiome, and when formulation ingredients are used without considering this, there is a problem in that there is a decrease not only in the effects of the active ingredients, but also in the general effects (moisturizing, soothing, and the like) of cosmetics.

With this background, the present inventors have made diligent efforts to identify formulation ingredients and compositions thereof that do not inhibit the growth of beneficial skin bacteria, and as a result, they have confirmed that the formulation ingredients do not inhibit the growth of beneficial skin bacteria and have beneficial skin bacteria-friendly properties in specific compositions, and applied the formulation ingredients to microbiome cosmetic compositions to complete the present invention.

### DISCLOSURE

### Technical Problem

An object of the present invention is to provide a cosmetic composition for maintaining or enhancing beneficial skin bacteria, which contains a surfactant and an oil.

Another object of the present invention is to provide a cosmetic composition for maintaining microbiome balance in the skin, which contains a surfactant and an oil.

Still another object of the present invention is to provide a method for preparing a cosmetic composition for maintaining or enhancing beneficial skin bacteria, which includes a) dissolving and dispersing a surfactant; b) preparing a mixture by adding an oil to the dispersed surfactant and performing mixing; and c) adding the mixture to water and emulsifying the mixture.

### Technical Solution

The present invention will be specifically described as follows. Meanwhile, each description and each embodiment disclosed in the present invention may also be applied to another description and another embodiment. In other words, all combinations of the various elements disclosed in the present invention fall within the scope of the present invention. In addition, it cannot be said that the scope of the present invention is limited to the specific description described below.

An aspect of the present invention provides a cosmetic composition for maintaining or enhancing beneficial skin bacteria, which contains a surfactant and an oil.

The term “beneficial skin bacteria” of the present invention refers to strains among skin flora which help improve skin defense by enhancing skin regeneration effects, which promote skin regeneration through cell membrane destruction (death) and growth suppression of harmful bacteria, which exhibit skin soothing and skin barrier strengthening effects by suppressing the expression of inflammatory factors, which exhibit the effect of increasing skin moisture content and suppressing moisture loss, and which maintain skin homeostasis by improving the pH environment by slightly acidifying the skin pH. The beneficial skin bacteria may be, for example, Staphylococcus epidermidis, but are not limited thereto.

The cosmetic composition of the present invention may have beneficial skin bacteria-friendly properties.

In the present invention, the beneficial skin bacteria-friendly properties may include maintaining beneficial skin bacteria and/or enhancing beneficial skin bacteria.

The term “maintaining beneficial skin bacteria” of the present invention means maintaining a certain population by assisting the growth of beneficial skin bacteria. For the purpose of the present invention, maintaining the beneficial skin bacteria may be maintaining Staphylococcus epidermidis, but is not limited thereto.

The term “enhancing beneficial skin bacteria” of the present invention means promoting or improving the growth of beneficial skin bacteria. For the purpose of the present invention, enhancing the beneficial skin bacteria may be enhancing Staphylococcus epidermidis, but is not limited thereto.

For example, in the present invention, a “bacterial growth index” parameter is used, and a composition with a bacterial growth index of −1.00 or more on day 1 after inoculation of bacteria is selected as a “cosmetic composition with beneficial skin bacteria-friendly properties”, which has the effect of maintaining beneficial skin bacteria and/or enhancing beneficial skin bacteria.

In other words, the cosmetic composition of the present invention may be a cosmetic composition containing a surfactant and an oil, which has a bacterial growth index of −1.00 or more on day 1 after inoculation of bacteria.

The surfactant of the present invention is not limited thereto, but may be, for example, a non-ionic surfactant, a phospholipid-based surfactant, and a non-phospholipid-based surfactant. In another example, the non-phospholipid-based surfactant may be sodium dilauramidoglutamide lysine, inulin lauryl carbamate, and the like, but is not limited thereto. For the purposes of the present invention, the surfactant may be a non-ionic surfactant, a phospholipid-based surfactant, and/or a non-phospholipid-based surfactant, but may include without limitation any surfactant that does not inhibit the growth of beneficial bacteria.

However, in the cosmetic composition of the present invention, surfactants belonging to anionic, cationic, and amphoteric surfactants other than the non-ionic, phospholipid-based, and non-phospholipid-based surfactants may be preferably contained at 1% or less based on the total weight of the cosmetic composition of the present invention, more preferably may be contained at 0.1% or less, and most preferably may be substantially not contained.

Examples of the anionic surfactant include cetyl phosphate, and specifically, potassium cetyl phosphate; cetyl phosphate, arginine; cetyl phosphate, sodium hydroxide; cetyl phosphate, tromethamine; sodium methyl stearoyl taurate; and sodium stearoyl glutamate, but are not limited thereto.

Examples of the cationic surfactant include, but are not limited to, distearyldimonium chloride and stearamidopropyl dimethylamine.

Examples of the amphoteric surfactant include, but are not limited to, lauryl hydroxysultaine and cocamidopropyl betaine.

In the present invention, the non-ionic surfactant may be, for example, a PEG (polyethylene glycol)-based non-ionic surfactant and/or a non-PEG-based non-ionic surfactant, but is not limited thereto.

The PEG-based non-ionic surfactant may be, for example, PEG-100 stearate, steareth-21, PEG-40 stearate, or PEG-60 hydrogenated castor oil, but is not limited thereto.

However, as the PEG-based non-ionic surfactant, for example, polysorbate 60, PEG-60 glyceryl isostearate, PEG-11 methyl ether dimethicone, PEG-10 dimethicone, or PEG-150 distearate may be preferably contained at 1% or less based on the total weight of the cosmetic composition of the present invention, more preferably may be contained at 0.1% or less, and most preferably may be substantially not contained.

The non-PEG-based non-ionic surfactant may be, for example, glyceryl stearate, cetearyl alcohol, cetearyl glucoside, fatty alcohols, alkyl glucosides, sorbitan olivate, cetearyl olivate, sorbitan stearate, sucrose polystearate, cetyl palmitate, polyglyceryl oleate, polyglyceryl laurate, or polyglyceryl stearate, but is not limited thereto.

The fatty alcohols may be, for example, lauryl alcohol, myristyl alcohol, cetyl alcohol, stearyl alcohol, arachidyl alcohol, behenyl alcohol, or C14-22 alcohols, but are not limited thereto.

The alkyl glucoside may be, for example, lauryl glucoside, myristyl glucoside, cetearyl glucoside, arachidyl glucoside, C12-18 alkyl glucoside, or C12-20 alkyl glucoside, but is not limited thereto.

The polyglyceryl oleate may be, for example, polyglyceryl-2 oleate, polyglyceryl-3 oleate, polyglyceryl-4 oleate, polyglyceryl-5 oleate, polyglyceryl-6 oleate, polyglyceryl-8 oleate, or polyglyceryl-10 oleate, but is not limited thereto.

The polyglyceryl laurate may be, for example, polyglyceryl-2 laurate, polyglyceryl-3 laurate, polyglyceryl-4 laurate, polyglyceryl-5 laurate, polyglyceryl-6 laurate, or polyglyceryl-10 laurate, but is not limited thereto.

The polyglyceryl stearate may be, for example, polyglyceryl-2 stearate, polyglyceryl-3 stearate, polyglyceryl-4 stearate, polyglyceryl-5 stearate, polyglyceryl-6 stearate, polyglyceryl-8 stearate, or polyglyceryl-10 stearate, but is not limited thereto.

However, as the non-PEG-based non-ionic surfactant, for example, coco-glucoside may be preferably contained at 1% or less based on the total weight of the cosmetic composition of the present invention, more preferably may be contained at 0.1% or less, and most preferably may be substantially not contained.

In the present invention, the phospholipid-based surfactant may be lecithin, hydrogenated lecithin, and the like, but is not limited thereto.

In the present invention, the non-phospholipid-based surfactant may be sodium dilauramidoglutamide lysine, inulin lauryl carbamate, and the like, and this is as described above.

The oil of the present invention may not be diethoxyethyl succinate, bis-ethoxydiglycol cyclohexane 1,4-dicarboxylate, heptyl undecylenate, Olea europaea (olive) fruit oil, or Persea gratissima (avocado) oil. Specifically, diethoxyethyl succinate, bis-ethoxydiglycol cyclohexane 1,4-dicarboxylate, heptyl undecylenate, Olea europaea (olive) fruit oil, Persea gratissima (avocado) oil, and the like may be preferably contained at 1% or less based on the total weight of the cosmetic composition of the present invention, more preferably may be contained at 0.1% or less, and most preferably may be substantially not contained. The oil of the present invention may include, without limitation, oils that do not inhibit the growth of beneficial bacteria, except for the above-mentioned oils.

For example, the oil of the present invention may be a hydrocarbon oil, an ester oil, a silicone oil, a natural oil, and the like, except for the above-mentioned oils, but is not limited thereto.

In the present invention, the hydrocarbon oil may be, for example, C13-15 alkane, hydrogenated polydecene, hydrogenated polyisobutene, isododecane, isohexadecane, squalane, undecane, tridecane, C13-14 alkane, C14-17 alkane, C14-19 alkane, or C15-19 alkane, but is not limited thereto.

In the present invention, the ester oil may be, for example, cetyl ethylhexanoate, hexyldecyl ethylhexanoate, isocetyl myristate, isotridecyl isononanoate, pentaerythrityl tetraisostearate, pentaerythrityl tetraethylhexanoate, triethylhexanoin, caprylic/capric triglyceride, coco-caprylate/caprate, dicaprylyl carbonate, phytosteryl/octyldodecyl lauroyl glutamate, trimethylolpropane tricaprylate/tricaprate, dipentaerythrityl hexa C5-9 acid esters, diisostearyl malate, hexyl laurate, neopentyl glycol diheptanoate, ethyl isostearate, isopropyl myristate, isostearyl isostearate, or octyldodecyl myristate, but is not limited thereto.

However, as the ester oil, for example, diethoxyethyl succinate, bis-ethoxydiglycol cyclohexane 1,4-dicarboxylate, or heptyl undecylenate may be preferably contained at 1% or less based on the total weight of the cosmetic composition of the present invention, more preferably may be contained at 0.1% or less, and most preferably may be substantially not contained. This is as described above.

In the present invention, the silicone oil may be, for example, caprylyl methicone, cyclohexasiloxane, cyclopentasiloxane, dimethiconol, dimethicone, methyl trimethicone, diphenyl dimethicone, diphenylsiloxy phenyl trimethicone, or phenyl trimethicone, but is not limited thereto.

In the present invention, the natural oil may be, for example, Helianthus annuus (sunflower) seed oil, Limnanthes alba (meadowfoam) seed oil, Macadamia ternifolia seed oil, Simmondsia chinensis (jojoba) seed oil, Avena sativa (oat) kernel oil, or Butyrospermum parkii (shea) butter, but is not limited thereto.

However, as the natural oil, for example, Olea europaea (olive) fruit oil or Persea gratissima (avocado) oil may be preferably contained at 1% or less based on the total weight of the cosmetic composition of the present invention, more preferably may be contained at 0.1% or less, and most preferably may be substantially not contained. This is as described above.

The cosmetic composition of the present invention may further contain a fatty acid.

In the present invention, the fatty acid may be myristic acid, stearic acid, or behenic acid, but fatty acids that do not inhibit the growth of beneficial bacteria may be contained without limitation.

In the cosmetic composition of the present invention, one or more ingredients of the oils and/or one or more ingredients of the fatty acids may be added.

The cosmetic composition of the present invention may be prepared into a formulation selected from the group consisting of solutions, external ointments, creams, foams, nutrient lotions, softening lotions, packs, softening water, milky lotions, makeup bases, essences, soaps, liquid cleansers, bath additives, sunscreen creams, sun oils, suspensions, emulsions, pastes, gels, lotions, powders, soaps, surfactant-containing cleansings, oils, powder foundations, emulsion foundations, wax foundations, patches, and sprays, but is not limited thereto.

In the present invention, the cosmetic composition may further contain one or more cosmetically acceptable carriers blended in general skin cosmetics, and for example, an oil ingredient, water, a surfactant, a humectant, a lower alcohol, a thickener, a chelating agent, a colorant, a preservative, and a flavoring agent may be appropriately blended as conventional ingredients, but the carrier is not limited thereto.

In an embodiment, the cosmetic composition of the present invention may further contain a thickening agent. The thickener may be, for example, acrylates/C10-30 alkyl acrylate crosspolymer, carbomer, polyacrylate crosspolymer-6, or xanthan gum, but is not limited thereto.

In another embodiment, the cosmetic composition of the present invention may further contain a vitamin. The vitamin may be, for example, panthenol, but is not limited thereto.

The cosmetically acceptable carriers contained in the cosmetic composition of the present invention vary depending on the formulation of the cosmetic composition.

In a case where the formulation of the present invention is ointments, pastes, creams or gels, animal oil, vegetable oil, wax, paraffin, starch, tragacanth, cellulose derivatives, polyethylene glycol, silicone, bentonite, silica, talc, zinc oxide, and the like may be used as a carrier ingredient, but the carrier ingredient is not limited thereto. These may be used singly or in a mixture of two or more kinds thereof.

In a case where the formulation of the present invention is powders or sprays, lactose, talc, silica, aluminum hydroxide, calcium silicate, polyamide powder, and the like may be used as a carrier ingredient. In particular, in a case where the formulation of the present invention is sprays, a propellant such as chlorofluorohydrocarbon, propane/butane, or dimethyl ether may be additionally contained, but the carrier ingredient is not limited thereto. These may be used singly or in a mixture of two or more kinds thereof.

In a case where the formulation of the present invention is solutions or emulsions, a solvent, a solubilizing agent, an emulsifying agent, or the like may be used as a carrier ingredient, and for example, water, ethanol, isopropanol, ethyl carbonate, ethyl acetate, benzyl alcohol, benzyl benzoate, propylene glycol, 1,3-butylene glycol, cottonseed oil, peanut oil, corn germ oil, castor oil, sesame oil, glycerol fatty acid esters, polyethylene glycol, or fatty acid esters of sorbitan may be used as a carrier ingredient, but the carrier ingredient is not limited thereto. These may be used singly or in a mixture of two or more kinds thereof.

In a case where the formulation of the present invention is suspensions, a liquid diluent such as water, ethanol, or propylene glycol, suspending agents such as ethoxylated isostearyl alcohols, polyoxyethylene sorbitol esters, and polyoxyethylene sorbitan esters, microcrystalline cellulose, aluminum metahydroxide, bentonite, agar, tragacanth, or the like may be used as a carrier ingredient, but the carrier ingredient is not limited thereto. These may be used singly or in a mixture of two or more kinds thereof.

In a case where the formulation of the present invention is soaps, alkali metal salts of fatty acids, fatty acid hemiester salts, fatty acid protein hydrolysates, isethionates, lanolin derivatives, fatty alcohols, vegetable oils, glycerol, sugar, and the like may be used as a carrier ingredient, but the carrier ingredient is not limited thereto. These may be used singly or in a mixture of two or more kinds thereof.

The cosmetic composition of the present invention may further contain water.

The cosmetic composition of the present invention may contain a surfactant and an oil at about 0.1% to 30% by weight, specifically at about 1% to 10% by weight, about 2% to 10% by weight, about 3% to 10% by weight, about 4% to 10% by weight, about 5% to 10% by weight, about 1% to 8% by weight, about 2% to 8% by weight, about 3% to 8% by weight, about 4% to 8% by weight, about 5% to 8% by weight, about 1% to 7% by weight, about 2% to 7% by weight, about 3% to 7% by weight, about 4% to 7% by weight, about 5% to 7% by weight, about 1% to 6% by weight, about 2% to 6% by weight, about 3% to 6% by weight, about 4% to 6% by weight, about 5% to 6% by weight, about 1% to 5% by weight, about 2% to 5% by weight, about 3% to 5% by weight, or about 4% to 5% by weight for each ingredient based on the total weight of the composition, but the content of each ingredient is not limited thereto.

The cosmetic composition of the present invention may further contain a fatty acid at about 0.1% to 30% by weight, specifically at about 1% to 10% by weight for each ingredient based on the total weight of the composition, but the content of each ingredient is not limited thereto.

In the present invention, the term “about” may be preceded by a specific numerical value. As used in this application, the term “about” includes not only the exact number that follows the term, but also a range that is nearly that number or is close to that number. It can be determined whether the number is close to or nearly the specific number mentioned, given the context in which it is presented. As an example, the term “about” may refer to a range of ˜10% to +10% of a numerical value. As another example, the term “about” may refer to a range of ˜5% to +5% of a given numerical value. However, the range is not limited thereto.

All ingredients described in the present invention preferably do not exceed the maximum use values stipulated in the Regulations on Cosmetics Safety Standards, and the like (Korean Regulations) and Cosmetics Safety Technical Regulations (Chinese Regulations).

Another aspect of the present invention provides a cosmetic composition for maintaining microbiome balance in the skin, which contains a surfactant and an oil.

Terms used here are as described above.

The composition of the present invention may further contain a fatty acid.

The composition of the present invention may further contain one or more selected from a thickener and a vitamin.

In the present invention, “microbiome” is a compound word of “microbiota” and “genome”, and means the entire microbiota and genome coexisting in humans, animals, plants, soils, the sea, lakes, rock walls, air, and the like. Among these, the microbiome in the skin is a group of microorganisms that form a complex interaction between microorganisms and microorganisms and between hosts and microorganisms existing in the skin, and refers to a part of the human microbiome.

“Maintaining microbiome balance in the skin” means properly maintaining the balance of microbiota in the skin by enhancing the activity and survival of beneficial bacteria in the skin while suppressing the proliferation and/or activity of harmful bacteria in the skin. Specifically, maintaining microbiome balance may be achieved by maintaining or enhancing beneficial skin bacteria. In the present invention, the beneficial skin bacteria may be Staphylococcus epidermidis.

Still another aspect of the present invention provides a method for preparing a cosmetic composition for maintaining or enhancing beneficial skin bacteria.

Specifically, the method may include a) dissolving and dispersing a surfactant; b) preparing a mixture by adding an oil to the dispersed surfactant and performing mixing; and c) adding the mixture to water and emulsifying the mixture.

Terms used here are as described above.

Step a) may include dissolving and dispersing a surfactant.

The dissolution may be performed at a high temperature, and for example, may be performed in a temperature range of 50° C. to 80° C. depending on the activation temperature of each surfactant, but is not limited thereto.

In an example, the surfactant may be non-ionic, phospholipid-based, and non-phospholipid-based surfactants, and in another example, the non-phospholipid-based surfactant may be sodium dilauramidoglutamide lysine, inulin lauryl carbamate, and the like, but is not limited thereto, and this is as described above.

Step b) may include preparing a mixture by adding an oil as an oil-soluble ingredient to the surfactant dispersed in step a) and performing mixing.

At this time, the surfactant and oil may be added at about 0.1% to 30% by weight, specifically at about 1% to 10% by weight, about 2% to 10% by weight, about 3% to 10% by weight, about 4% to 10% by weight, about 5% to 10% by weight, about 1% to 8% by weight, about 2% to 8% by weight, about 3% to 8% by weight, about 4% to 8% by weight, about 5% to 8% by weight, about 1% to 7% by weight, about 2% to 7% by weight, about 3% to 7% by weight, about 4% to 7% by weight, about 5% to 7% by weight, about 1% to 6% by weight, about 2% to 6% by weight, about 3% to 6% by weight, about 4% to 6% by weight, about 5% to 6% by weight, about 1% to 5% by weight, about 2% to 5% by weight, about 3% to 5% by weight, or about 4% to 5% by weight for each ingredient, but the content of each ingredient is not limited thereto.

The oil may not be diethoxyethyl succinate, bis-ethoxydiglycol cyclohexane 1,4-dicarboxylate, heptyl undecylenate, Olea europaea (olive) fruit oil, or Persea gratissima (avocado) oil. The oil of the present invention may include, without limitation, oils that do not inhibit the growth of beneficial bacteria, except for the above-mentioned oils.

For example, the oil of the present invention may be a hydrocarbon oil, an ester oil, a silicone oil, a natural oil, and the like, except for the above-mentioned oils, but is not limited thereto, and this is as described above.

In step b), a fatty acid as an oil-soluble ingredient may be additionally added, and mixing may be performed, but step b) is not limited thereto.

The fatty acid may be myristic acid, stearic acid, behenic acid, and the like, but may include fatty acids that do not inhibit the growth of beneficial bacteria without limitation, and this is as described above.

In step b), one or more ingredients of the oils and/or one or more ingredients of the fatty acids may be added.

Step c) may include adding the mixture of step b) to water and emulsifying the mixture.

Specifically, the mixture may be gradually added to water so that the total percentage by weight becomes 100% by weight, and then emulsified for about 1 to 10 minutes using a homogenizer, but step c) is not limited thereto.

In step b) or c), any one or more selected from a thickener or a vitamin may be further added and emulsification may be performed, but step b) or c) is not limited thereto.

The method may further include cooling and discharging the emulsified mixture after step c), but is not limited thereto.

The cosmetic composition prepared by the method of the present invention may have beneficial skin bacteria-friendly properties including an effect of maintaining or enhancing beneficial skin bacteria, and may have an effect of maintaining microbiome balance in the skin.

Still another aspect of the present invention provides a method for maintaining or enhancing beneficial skin bacteria, which includes applying a cosmetic composition containing a surfactant and an oil to the skin of a subject.

As used herein, the term “applying” or “administering” means introducing the composition of the present invention to a subject by any suitable method, and the application route of the composition may be any general route as long as the composition can reach the target tissue therethrough. Due to the nature of the composition of the present invention having an effect of maintaining or enhancing beneficial skin bacteria, the application route of the composition may be applied by applying it to the skin.

The composition of the present invention may be applied daily or intermittently, and may be applied once daily or divided into 2 or 3 applications. In addition, the composition of the present invention may be used alone or in combination with other drug treatments to maintain or enhance beneficial skin bacteria. It is important to apply the amount that can obtain the maximum effect with the minimum amount without side effects in consideration of all of the above factors, and the dose can easily be determined by those skilled in the art.

As used herein, the term “a subject” refers to all animals, such as rats, mice, and livestock, including humans, that require maintenance of microbiome balance in the skin, specifically maintenance or enhancement of beneficial skin bacteria. Specifically, the subject may be a mammal including humans.

Still another aspect of the present invention provides a method for maintaining the microbiome balance in the skin, which includes applying a cosmetic composition containing a surfactant and an oil to the skin of a subject.

The terms used herein are as described above.

### Advantageous Effects

The composition of the present invention does not inhibit the growth of beneficial skin bacteria, has beneficial skin bacteria-friendly properties, can maintain or promote the growth of beneficial bacteria without addition of active materials such as prebiotics or probiotics, and therefore can be provided as a microbiome cosmetic composition.

### BRIEF DESCRIPTION OF DRAWINGS

FIG. 1 is a schematic diagram of a method for preparing a composition for evaluating beneficial skin bacteria affinity;

FIG. 2 is a diagram illustrating the bacterial growth index according to the beneficial skin bacteria affinity evaluation of each surfactant;

FIG. 3 is a diagram illustrating the bacterial growth index according to the beneficial skin bacteria affinity evaluation of each oil-soluble ingredient (oil); and

FIG. 4 is a diagram illustrating the beneficial skin bacteria affinity evaluation results of cosmetic compositions of Example 1 and Comparative Examples 1 to 3.

### DETAILED DESCRIPTION OF THE INVENTION

Hereinafter, the present invention will be described in more detail with reference to Examples. However, these Examples are intended to illustrate the present invention by way of example, and the scope of the present invention is not limited by these Examples, and it will be clear to those skilled in the art to which the present invention pertains.

### Preparation Example 1. Preparation of Composition for Evaluating Beneficial Skin Bacteria Affinity

In order to evaluate the beneficial skin bacteria affinity of surfactants and oil-soluble ingredients among formulation ingredients, a composition was prepared as an oil-in-water dispersed phase as follows.

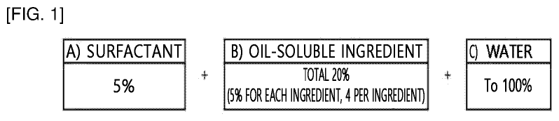

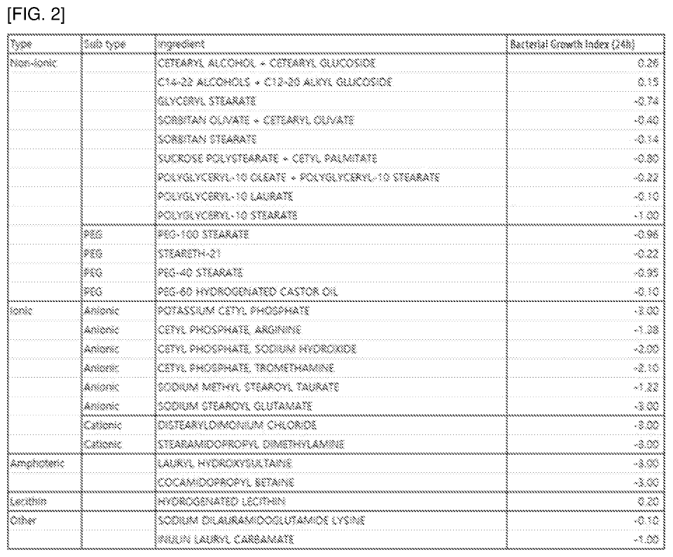

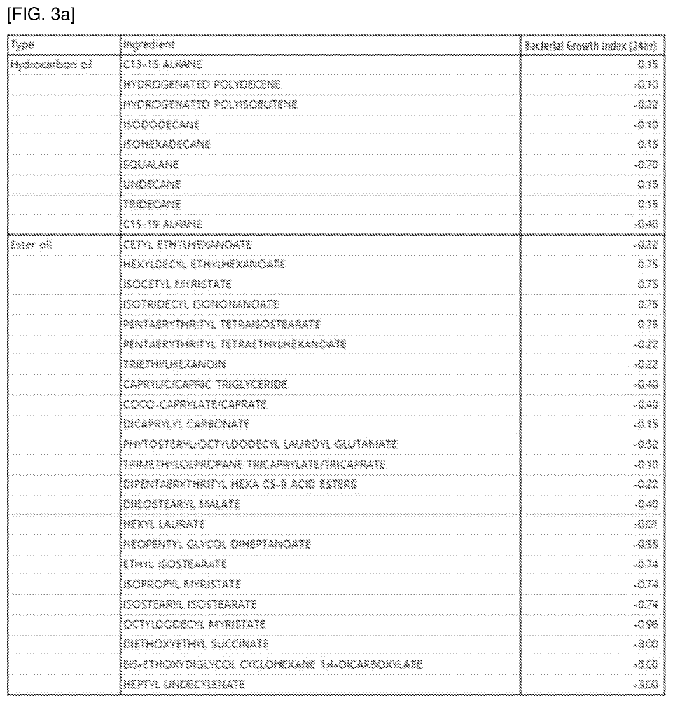

Specifically, as illustrated in FIG. 2, one type of surfactant was selected from among a total of seven types of surfactants [non-ionic (PEG (polyethylene glycol) and non-PEG) surfactants, ionic (anionic, cationic, and amphoteric) surfactants, and other (phospholipid and non-phospholipid) surfactants] classified according to functional groups, weighed, and then dissolved and dispersed in a temperature range of 50° C. to 80° C. depending on the activation temperature of each surfactant. The oil-soluble ingredient (oil) was selected from among a total of four types of oils [hydrocarbon, ester, silicone, and natural (naturally derived) oils] classified according to functional groups as illustrated in FIG. 3, and up to 4 ingredients, weighed to be 5% by weight (maximum 20% by weight in the present invention) for each ingredient, was mixed with the surfactant, and stirring was performed until the mixture became uniform. The mixture was gradually added to water so that the total percentage by weight became 100% by weight, emulsified for about 5 minutes using a homogenizer, cooled, and then discharged (FIG. 1).

### Experimental Example 1. Beneficial Skin Bacteria Affinity Evaluation

As the beneficial bacteria serving as the affinity evaluation criterion of each composition prepared in Preparation Example 1, Staphylococcus epidermidis (S. epidermidis), a Gram positive bacterium, was selected. S. epidermidis is part of the skin flora present in large numbers in healthy skin, and is a representative beneficial bacterium that helps improve skin immunity and protect skin from external environmental factors by producing peptide antibiotics or PSM (phenol-soluble modulins) (Parlet, C. P., et al. (2019) Trends in microbiology, 27(6), 497-507). The evaluation of affinity for beneficial bacteria was performed by the challenge test method (Russell A. D., (2003), Int J Cosmet Sci., 25(3), 147-153) as follows.

Specifically, S. epidermidis was first incubated in TSA (Tryptic Soy Agar) medium at 37° C. for 24 hours, and then inoculated into 50.0 g of each composition prepared in Preparation Example 1 so as to be 1×10⁶ CFU to 1×10⁷ CFU (colony-forming units)/mL. After inoculation, the bacteria were evenly mixed in the composition by vortexing or using a sterilized stick. After inoculation, the mixture was stored at room temperature. In consideration of the product usage cycle, such as cosmetics, and the general usage patterns of these products, a maximum of 24 hours was set as the time required for examination. With this determination, 24 hours after inoculation, 1 mL of the composition inoculated with the bacteria was diluted stepwise using a 0.85% NaCl solution, 100 μL of each diluted solution was spread on TSA medium, incubation was conducted at 37° C. for 48 hours, and then CFUs were counted.

As the affinity evaluation criterion, the following “bacterial growth index*” parameter was used, and a composition having a bacterial growth index of −1.00 or more on day 1 after inoculation of bacteria was selected as a “cosmetic with beneficial skin bacteria-friendly properties” having an effect of maintaining beneficial skin bacteria or enhancing beneficial skin bacteria.

*Bacterial growth index=log₁₀[{Number of viable bacteria (CFU/g)}/{Number of inoculated bacteria (CFU/g)}]

As a result, as illustrated in FIG. 2, it was confirmed that ionic (anionic, cationic, and amphoteric) surfactants and a plurality of PEG-containing surfactants rapidly inhibit the growth of beneficial bacteria. On the other hand, non-ionic surfactants, specifically PEG-based or non-PEG-based surfactants, phospholipid-based surfactants, and non-phospholipid-based surfactants (sodium dilauramidoglutamide lysine and inulin lauryl carbamate) did not inhibit the growth of beneficial bacteria.

In particular, from the results, it was confirmed that phospholipid-based surfactants have a remarkable effect of maintaining or enhancing beneficial bacteria, and thus it can be concluded that phospholipid-based surfactants are suitable for a cosmetic composition with beneficial skin bacteria-friendly properties.

As illustrated in FIG. 3, it was confirmed that among the oil-soluble ingredients, oils such as diethoxyethyl succinate, bis-ethoxydiglycol cyclohexane 1,4-dicarboxylate, heptyl undecylenate, Olea europaea (olive) fruit oil, or Persea gratissima (avocado) oil inhibit the growth of beneficial bacteria. On the other hand, it was confirmed that hydrocarbon oils and silicone oils do not inhibit the growth of beneficial bacteria and maintain the bacterial growth index of −1.0 or more until day 1 or day 2. Most ester oils and natural oils do not inhibit the growth of beneficial bacteria as the bacterial growth index exceeds −1.0, although there is a slight difference in degree.

### Preparation Example 2. Preparation of Cosmetic Composition with Beneficial Skin Bacteria-Friendly Properties

Based on the results of Experimental Example 1, a cosmetic composition containing a surfactant and an oil-soluble ingredient, which were confirmed not to inhibit the growth of beneficial bacteria, was prepared by the method of Preparation Example 1. The ingredients for Example 1 and Comparative Examples 1 to 3 are shown in the following Table 1.

TABLE 1

| | | | Comparative | Comparative | Comparative |
|---|---|---|---|---|---|
| | INCI | Example 1 | Example 1 | Example 2 | Example 3 |
| A) Surfactant | C14-22 ALCOHOLS | 2.50 | | | |
| | C12-20 ALKYL GLUCOSIDE | | | | |
| | HYDROGENATED LECITHIN | 2.50 | | | |
| | PEG-60 GLYCERYL ISOSTEARATE | | 5.00 | | 2.50 |
| | POTASSIUM CETYL PHOSPHATE | | | 5.00 | 2.50 |
| B) Oil-soluble | HYDROGENATED POLYDECENE | 5.00 | | | |
| ingredient | ISODODECANE | 5.00 | | | |
| | UNDECANE | 5.00 | | | |
| | TRIDECANE | | | | |
| | HYDROGENATED | 5.00 | | | |
| | POLYISOBUTENE | | | | |
| | OLEA EUROPAEA (OLIVE) FRUIT | | 5.00 | 5.00 | 5.00 |
| | OIL | | | | |
| | DIETHOXYETHYL SUCCINATE | | 5.00 | 5.00 | 5.00 |
| | BIS-ETHOXYDIGLYCOL | | 5.00 | 5.00 | 5.00 |
| | CYCLOHEXANE | | | | |
| | 1,4-DICARBOXYLATE | | | | |
| | HEPTYL UNDECYLENATE | | 5.00 | 5.00 | 5.00 |
| C) Water and | TROMETHAMINE | 0.80 | 0.80 | 0.80 | 0.80 |
| others | ACRYLATES/C10-30 ALKYL | 0.10 | 0.10 | 0.10 | 0.10 |
| | ACRYLATE CROSSPOLYMER | | | | |
| | XANTHAN GUM | 0.10 | 0.10 | 0.10 | 0.10 |
| | GLYCERIN | 8.50 | 8.50 | 8.50 | 8.50 |
| | DIPROPYLENE GLYCOL | 4.00 | 4.00 | 4.00 | 4.00 |
| | PANTHENOL | 2.00 | 2.00 | 2.00 | 2.00 |
| | WATER | To 100 | To 100 | To 100 | To 100 |

### Experimental Example 2. Beneficial Skin Bacteria Affinity Evaluation of Example 1 and Comparative Examples 1 to 3

The beneficial skin bacteria affinity evaluation of the cosmetic compositions of Example 1 and Comparative Examples 1 to 3 prepared in Preparation Example 2 was performed by way of the method of Experimental Example 1, and the results are shown in Tables 2 and 3 below.

TABLE 2

| | | Comparative | Comparative | Comparative |
|---|---|---|---|---|
| CFU/g | Example 1 | Example 1 | Example 2 | Example 3 |
| Day 0 | 10,360,000 | 10,360,000 | 10,360,000 | 10,360,000 |
| Day 1 | 1,300,000 | 1,000 | 54,000 | 10,000 |

TABLE 3

| Bacterial growth | | Comparative | Comparative | Comparative |
|---|---|---|---|---|
| index | Example 1 | Example 1 | Example 2 | Example 3 |
| Day 0 | 0 | 0 | 0 | 0 |
| Day 1 | −0.90 | −4.02 | −2.28 | −3.02 |

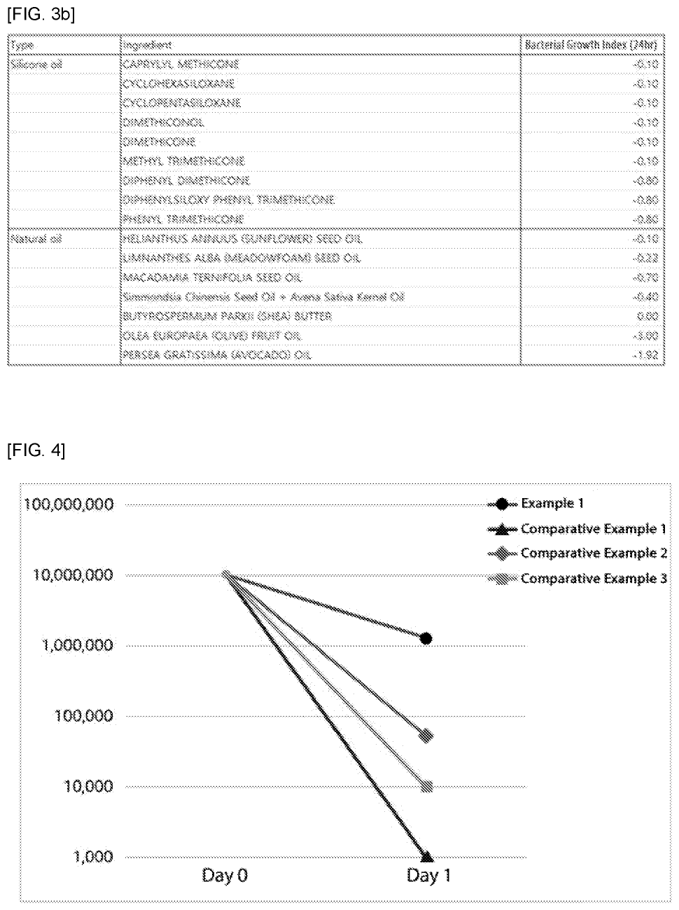

As shown in Tables 2 and 3, the formulation of Example 1 comprising hydrocarbon oils and non-ionic and phospholipid surfactants, which have beneficial bacteria-friendly properties, does not inhibit the growth of S. epidermidis, and the formulations of Comparative Examples 1 to 3 inhibit the growth of S. epidermidis (FIG. 4).

As it can be confirmed that Example 1 does not inhibit the growth of beneficial skin bacteria, has beneficial skin bacteria-friendly properties, and can maintain or promote the growth of beneficial bacteria without the addition of active materials such as prebiotics or probiotics, it can be concluded that the formulation can be provided as a microbiome cosmetic composition.

From the above description, those skilled in the art to which the present invention pertains will be able to understand that the present invention may be embodied in other specific forms without changing the technical spirit or essential characteristics thereof. In this regard, it should be understood that the embodiments described above are illustrative in all respects and not limiting. The scope of the present invention should be construed as including all changes or modifications derived from the meaning and scope of the following claims and their equivalent concepts rather than the detailed description above.

1. A method for maintaining or enhancing beneficial skin bacteria, the method comprising applying a cosmetic composition comprising a surfactant and an oil to the skin of a subject.

2. The method according to claim 1, wherein the cosmetic composition further comprises a fatty acid.

3. The method according to claim 1, wherein the beneficial skin bacteria are Staphylococcus epidermidis.

4. The method according to claim 1, wherein the surfactant is any one or more selected from the group consisting of a non-ionic surfactant, a phospholipid-based surfactant, and a non-phospholipid-based surfactant;
wherein the non-phospholipid-based surfactant is any one or more selected from the group consisting of sodium dilauramidoglutamide lysine and inulin lauryl carbamate;
wherein the non-ionic surfactant is any one or more selected from the group consisting of a polyethylene glycol (PEG)-based non-ionic surfactant and a non-PEG-based non-ionic surfactant; and
wherein the non-phospholipid-based surfactant is lecithin or hydrogenated lecithin.

5. The method according to claim 4, wherein the PEG-based non-ionic surfactant is any one or more selected from the group consisting of PEG-100 stearate, Steareth-21, PEG-40 stearate, and PEG-60 hydrogenated castor oil; and
the non-PEG-based non-ionic surfactant is any one or more selected from the group consisting of glyceryl stearate, cetearyl alcohol, cetearyl glucoside, lauryl alcohol, myristyl alcohol, cetyl alcohol, stearyl alcohol, arachidyl alcohol, behenyl alcohol, C14-22 alcohol, lauryl glucoside, myristyl glucoside, cetearyl glucoside, arachidyl glucoside, C12-18 alkyl glucoside, C12-20 alkyl glucoside, sorbitan olivate, cetearyl olivate, sorbitan stearate, sucrose polystearate, cetyl palmitate, polyglyceryl-2 oleate, polyglyceryl-3 oleate, polyglyceryl-4 oleate, polyglyceryl-5 oleate, polyglyceryl-6 oleate, polyglyceryl-8 oleate, polyglyceryl-10 oleate, polyglyceryl-2 laurate, polyglyceryl-3 laurate, polyglyceryl-4 laurate, polyglyceryl-5 laurate, polyglyceryl-6 laurate, polyglyceryl-10 laurate, polyglyceryl-2 stearate, polyglyceryl-3 stearate, polyglyceryl-4 stearate, polyglyceryl-5 stearate, polyglyceryl-6 stearate, polyglyceryl-8 stearate, and polyglyceryl-10 stearate.

6. The method according to claim 1, wherein the oil is any one or more selected from the group consisting of hydrocarbon oil, ester oil, silicone oil, and natural oil.

7. The method according to claim 6, wherein the hydrocarbon oil is any one or more selected from the group consisting of C13-15 alkane, hydrogenated polydecene, hydrogenated polyisobutene, isododecane, isohexadecane, squalane, undecane, tridecane, C13-14 alkane, C14-17 alkane, C14-19 alkane, and C15-19 alkane;
the ester oil is any one or more selected from the group consisting of cetyl ethylhexanoate, hexyldecyl ethylhexanoate, isocetyl myristate, isotridecyl isononanoate, pentaerythrityl tetraisostearate, pentaerythrityl tetraethylhexanoate, triethylhexanoin, caprylic/capric triglyceride, coco-caprylate/caprate, dicaprylyl carbonate, phytosteryl/octyldodecyl lauroyl glutamate, trimethylolpropane tricaprylate/tricaprate, dipentaerythrityl hexa C5-9 acid esters, diisostearyl malate, hexyl laurate, neopentyl glycol diheptanoate, ethyl ethyl isostearate, isopropyl myristate, isostearyl isostearate, and octyldodecyl myristate;
the silicone oil is any one or more selected from the group consisting of caprylyl methicone, cyclohexasiloxane, cyclopentasiloxane, dimethiconol, dimethicone, methyl trimethicone, diphenyl dimethicone, diphenylsiloxy phenyl trimethicone, and phenyl trimethicone; and
the natural oil is any one or more selected from the group consisting of Helianthus annuus (sunflower) seed oil, Limnanthes alba (meadowfoam) seed oil, Macadamia ternifolia seed oil, Simmondsia chinensis (jojoba) seed oil, Avena sativa (oat) kernel oil, and Butyrospermum parkii (shea) butter.

8. The method according to claim 6, wherein the ester oil is not diethoxyethyl succinate, bis-ethoxydiglycol cyclohexane 1,4-dicarboxylate, or heptyl undecylenate.

9. The method according to claim 6, wherein the natural oil is not Olea europaea (OLIVE) fruit oil or Persea gratissima (avocado) oil.

10. The method according to claim 2, wherein the fatty acid is any one or more selected from the group consisting of myristic acid, stearic acid, and behenic acid.

11. The method according to claim 1, wherein the cosmetic composition further comprises any one or more selected from the group consisting of a thickener and a vitamin.

12. The method according to claim 11, wherein the thickener is any one or more selected from the group consisting of acrylates/C10-30 alkyl acrylate crosspolymer, carbomer, polyacrylate crosspolymer-6, and xanthan gum; and
the vitamin is panthenol.

13. The method according to claim 1, wherein the cosmetic composition comprises a surfactant and an oil at 1% to 10% by weight for each ingredient based on a total weight of the cosmetic composition.

14. A method for maintaining the microbiome balance in the skin, the method comprising applying a cosmetic composition comprising a surfactant and an oil to the skin of a subject.

15. The method according to claim 14, wherein the cosmetic composition further comprises a fatty acid.

16. The method according to claim 14, wherein maintaining microbiome balance in the skin is achieved by maintaining or enhancing beneficial skin bacteria.

17. The method according to claim 16, wherein the beneficial skin bacteria are Staphylococcus epidermidis.
